(19) United States
(12) Patent Application Publication
(10) Pub. No.: US 2010/0160872 A1
(43) Pub. Date: Jun. 24, 2010

(54) Eye-Guard

(76) Inventor: Ian Harrison, Dyfed (GB)

Correspondence Address: GORDON & JACOBSON, P.C., 60 LONG RIDGE ROAD, SUITE 407, STAMFORD, CT, 06902, US

(21) Appl. No.: 12/094,444

(22) PCT Filed: Aug. 3, 2006
(86) PCT No.: PCT/GB2006/002895

§ 371 Date: Oct. 6, 2009

(30) Foreign Application Priority Data
Aug. 5, 2005 (GB) 0516106.2
Feb. 17, 2006 (GB) 0603205.6

(51) Int. Cl. A61F 9/00; A61M 35/00

(52) U.S. Cl. 604/298; 604/302

(57) ABSTRACT

The present invention relates to medication applied to the eye, and in particular to an apparatus for applying such medication. An arrangement as described herein enables precise quantities of medication to be administered without the apparatus contacting the eye ball. The application comprises a shield portion (12) for application over a user's eye, comprising an opening (20, 22) for receiving a support into which medication can be dispensed. The opening is configured to guide and release the medication from the support into the user's eye.

### BACKGROUND OF THE INVENTION

1. Field of the Invention

The present invention relates generally to medication applied to the eye, and more specifically to apparatus for applying medication to the eye.

2. State of the Art

Medication in the form of eye drops is commonly used to treat many ocular ailments, wherein the patient is supplied with eye drops to be administered directly into the eye at regular intervals.

Providing the patient is of a reasonable age and the treatment only requires a quantity of the medication to be administered to the eye in general, this task is not so much of a problem. However, if the patient is infirm for example, and/or if it is required to administer a precise quantity of the medication to a specific area of the eye, this task can become significantly more difficult and demanding. In addition, contact between the dispenser and the eyeball should be avoided at all costs to avoid the possibility of infection or injury.

### SUMMARY OF THE INVENTION

The present invention to provide an applicator for enabling liquid medication to be safely and conveniently applied to the correct part of the eye without the possibility of contact between the eye and the dispenser from which said liquid medication is dispensed.

In accordance with the present invention, there is provided an applicator for applying liquid medication to a user's eye, the applicator comprising a shield portion for application over said user's eye, said shield portion comprising an opening for receiving a support into which said medication can be dispensed, wherein said opening is configured to guide and release said medication from said support into said user's eye.

Thus, the above-mentioned object is achieved by providing an applicator, that can be held over a user's eye, thereby shielding the eye, and whereby the eye drops can be administered very precisely via the support and opening in the shield, without the possibility of contact between the eye and the medication dispenser.

In a preferred embodiment, the support is adjustably mounted in the opening such that it's location relative to the user's eye can be selectively adjusted. In one exemplary embodiment, the opening may comprise one or more elongate channels in which the support can be slidably mounted. Means may be provided for immobilizing the support at a desired position within said opening. Such means may comprise means for generating mutual friction between said support and a contact edge of said opening.

In one exemplary embodiment, the opening may be provided in a plate portion, rotatably mounted in or on said shield portion, such that said opening can be rotated with respect to a generally central, substantially vertical axis, allowing location of the support relative to the users eye at any point within a circle swept by the length of the opening to be achieved.

Beneficially, at least one retaining strap is provided for retaining said shield portion over said user's eye, said retaining strap beneficially being connected to the applicator at substantially diametrically opposing edge parts thereof. The strap may comprise an elastic retaining strap, fashioned as a single piece connected at each side of the shield portion or in two pieces with a means for releasably joining the two.

Preferably, said shield portion is comprised of a rigid or semi-rigid transparent material.

Beneficially, a rim is provided on said shield portion, configured to facilitate a comfortable interface between said applicator and the socket around said user's eye.

Preferably, said opening comprises two elongate channels in communication with each other; the channels beneficially intersect, possibly at respective mid-points thereof. The channels may be substantially orthogonal relative to each other.

Beneficially, said support has a neck portion that is suitably received in said opening. The neck portion is beneficially provided with an outer circumferential groove with which the contact edges of the opening engage.

At least one of the channels preferably further comprises an enlarged opening therein. The enlarged opening of the channel is preferably located at an end of a channel, and is shaped and configured to enable the outer circumferential groove of said support to pass therethrough. This enables cleaning of the support, and provides a further advantage that the applicator may be worn at night for example. The shape of the circumferential groove preferably matches that of the enlarged opening such that the groove must be at the same configuration as the opening to release therefrom.

Preferably, an opening in said support contains means is provided, such as helical wall parts, to regulate the flow of medication passing from said support to said user's eye.

Beneficially, said support is generally frusto-conical in shape, being beneficially tapered inwardly from an upper opening to a lower neck portion.

These and other aspects of the present invention will be apparent from, and elucidated with reference to, the embodiments described herein.

Embodiments of the present invention will now be described, by way of example only, and with reference to the accompanying drawings.

### BRIEF DESCRIPTION OF THE DRAWINGS

FIG. 1 is a plan view of an applicator according to a first exemplary embodiment of the present invention;

FIG. 2 is a side view of the applicator of FIG. 1;

FIG. 3 is a plan view of an applicator according to a second exemplary embodiment of the present invention; and

FIG. 4 is a side view of the applicator of FIG. 3.

### BRIEF DESCRIPTION OF THE PREFERRED EMBODIMENTS

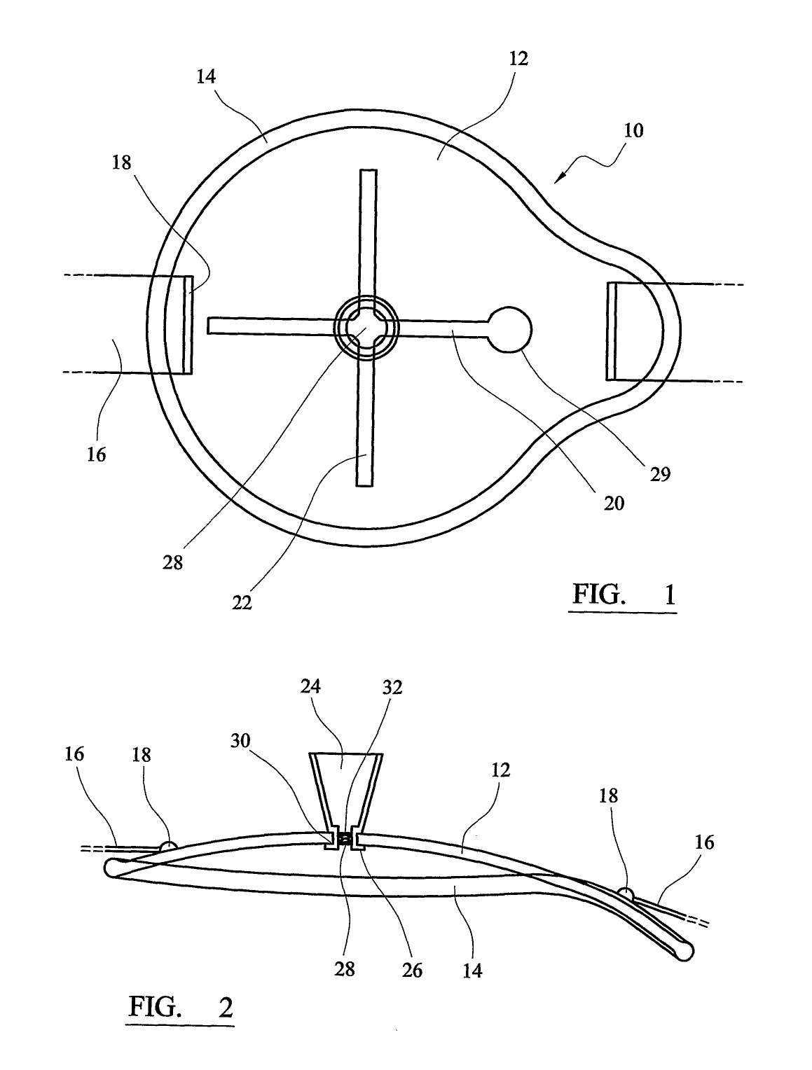

Referring to FIGS. 1 and 2 of the accompanying drawings, a first exemplary embodiment of an applicator 10 according to the invention comprises a generally convex shield portion 12 shaped and configured in accordance with the contours of a human eye socket such that, when the applicator 10 is positioned for use over an eye, the rim 14 sits comfortably within the recess defined by the eye socket. The rim 14 may be integrally moulded with the shield portion 12, or it may comprise a separate piece of the same or a different material, that is adhered to the perimeter of the shield portion 12. The shield portion 12 is preferably made of a rigid or semi-rigid, preferably transparent material such as acrylic, such that a user can see through it, at least to a certain extent, when the applicator 10 is positioned for use over the eye.

An elasticated retaining strap 16 is connected to the applicator 10, at diametrically opposing edge parts thereof, by respective fastening sections 18, that are anchored to the shield portion 12. This retaining strap 16 allows a user to secure the applicator 10 over the eye by engaging the strap 16, possibly around the back of the head, leaving both the users hands free.

The convex shield portion 12 defines two elongate, intersecting, orthogonal openings 20, 22 through its surface at an essentially central position, defining a substantially cross-shaped channel. A generally circular aperture 28 is defined at the point of intersection of the openings 20, 22.

A support 24 is retained in, and extends upwardly from, the openings 20, 22, such that it is capable of being slidably moved to any point along the channel 20, 22. The support 24 is generally conical, inwardly tapered from the upper opening to a lower neck portion. The neck portion defines an outer circumferential groove within which the edges of the channel 20, 22 are retained, the groove being defined between the lower and of the conical portion of the support 24 and a lower lip 26, which retains the support in place in the channel 20, 22 but does not restrict its lateral movement along it.

The outer circumferential groove provided at the neck portion 30 of the support 24 may be provided with a rubber or similar outer covering, such that relative movement between the neck portion 30 and the contact edges of the channel 20, 22, will require a reasonable amount of force to effect and when the support 24 is positioned in a desirable position within the channel 20, 22 it will be retained there by mutual friction unless it is manually moved with sufficient force to overcome the friction.

The function of the support 24 is to receive the dispensing nozzle of an eye-drop container and guide the medication dispensed therefrom through, via an aperture 28, onto the users eyes. As such, the position of the support 24 within the channel defined by the intersecting, elongate openings 20, 22 dictates the location on an eye that a drop of medication will fall, whilst ensuring that the eye-drop container can never contact the eyeball itself.

The aperture 28 that allows the drop to pass defines a passageway that regulates the flow of liquid passing through it and is shaped and configured to reduce ‘splash back’. A suitable structure 13 helical wall parts 32 enabling liquid to pass therethrough but preventing accidental damage to the edge by the dispensing nozzle of the eye drop container or the liquid contacting the eyeball at excess velocity.

The channels 20, 22 further define a widened portion 29 at an end of one of the openings, and as an exemplary embodiment having sufficient diameter to enable the lower lip 26 to pass therethrough. Although in use this is not desirable, in certain circumstances this may be extremely beneficial. For example, when the support 24 needs to be cleaned, it may be washed out without soaking the entire applicator 10. Additionally, if different eye medications are required, then there is a reduced chance of contamination due to the ease of cleaning. A second advantage of the inclusion of this feature is that the applicator 10 may also be used as an eye guard for a user to sleep in for example, without fear that the support 24 may become tangled in bed.

In a preferred embodiment, the widened portion is shaped to correspond to the shape of the lower lip 26 such that the support 24 must be rotated to the correct rotation that enables the support to release from the widened portion 29, which prevents accidental release.

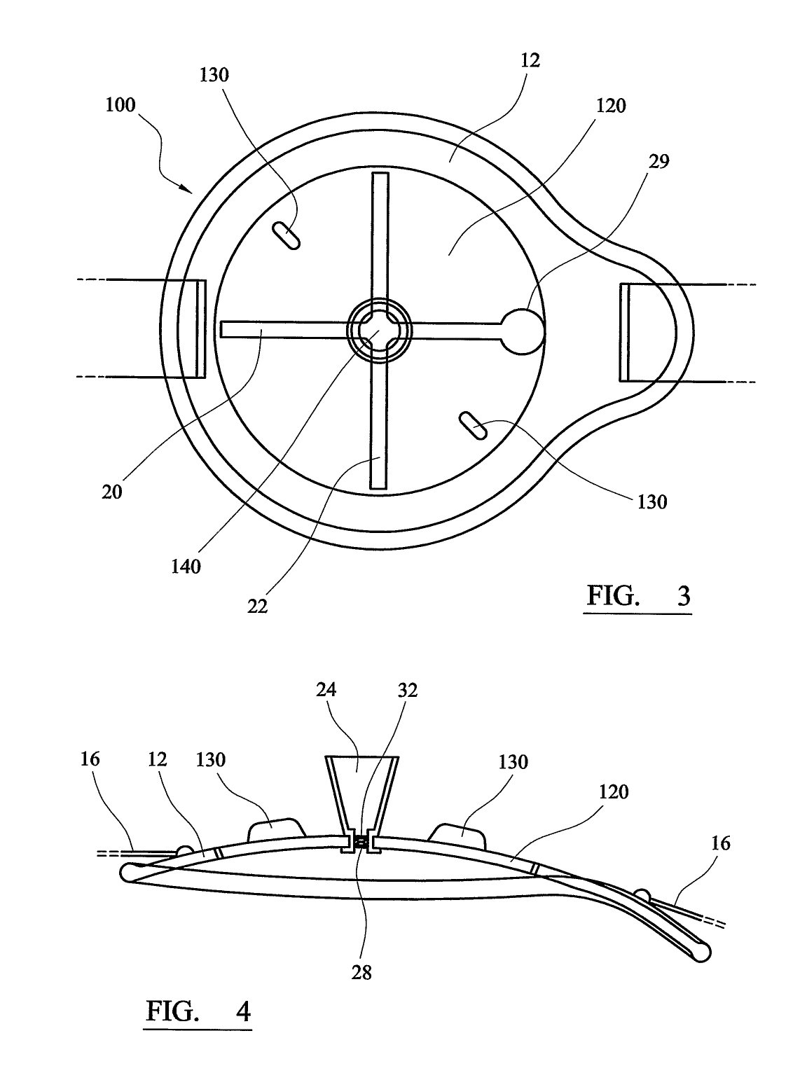

Referring now to FIGS. 3 and 4, an applicator 100 according to a second exemplary embodiment of the present invention is similar in many respects to that of the first exemplary embodiment, as defined above, but with means for rotating the channels 200, 220 about their central axis (being the centre-point of intersection), 140. The rotating means comprises a generally circular section 120b of the shield portion 120 being rotatably mounted on the main body of the shield portion 120, such that it can be manually rotated. A number of small raised portions 130 may be provided on the circular section 120 to facilitate convenient manual rotation thereof. These raised portions 130 may be integrally moulded or fixed to the rotatably mounted circular section 120.

The substantially cross shaped channel 200, 220 is formed in the rotatably mounted circular section 120 and as such, any applicable point on the users eyeball, within the swept circumference defined by the end points of the channels 200, 220 (neglecting the radius of the section of the support 240 that passes through the channel 200, 220) can be attained by moving the support 240 along the channel 200, 220, then rotating the rotatably mounted circular section 120. It will be appreciated that it is not essential to have such a configuration of channels 200, 220 as an equally satisfactory result could be attained by, for example, a single channel defining a radius of the rotatably mounted circular section 120, or any other suitable configuration.

It should be noted that the above-mentioned embodiment illustrates rather than limits the invention, and that those skilled in the art will be capable of designing many alternative embodiments without departing from the scope of the invention as defined by the appended claims. In the claims, any reference signs placed in parentheses shall not be construed as limiting the claims. The word “comprising” and “comprises”, and the like, do not exclude the presence of elements or steps other than those listed in any claim or the specification as a whole. The singular reference of an element does not exclude the plural reference of such elements and vice-versa. The invention may be implemented by means of hardware comprising several distinct elements. In a device claim enumerating several means, several of these means may be embodied by one and the same item of hardware. The mere fact that certain measures are recited in mutually different dependent claims does not indicate that a combination of these measures cannot be used to advantage.

1. An applicator for applying liquid medication to a user's eye, the applicator comprising a shield portion for application over said user's eye, said shield portion comprising an opening for receiving a support into which said medication can be dispensed, wherein said opening is configured to guide and release said medication from said support into said user's eye.

2. An applicator according to claim 1, wherein said support is adjustably mounted in said opening such that its location relative to the user's eye can be selectively adjusted.

3. An applicator according to claim 1, wherein said opening may comprise at least one elongate channel in which the support can be slidably mounted.

4. An applicator according to claim 1, wherein first means is provided for immobilizing the support at a desired position within said opening.

5. An applicator according to claim 4, wherein said first means comprises means for generating mutual friction between said support and a contact edge of said opening.

6. An applicator according to claim 1, wherein said opening is defined by a plate portion, rotatably mounted in or on said shield portion, such that said opening can be rotated with respect to a generally central, substantially vertical axis, allowing location of the support relative to the user's eye at any point within a circle swept by the length of the opening.

7. An applicator according to claim 1, wherein at least one retaining strap is provided for retaining said shield portion over said user's eye, said retaining strap being connected to the applicator at substantially diametrically opposing edges thereof.

8. An applicator according to claim 7, wherein said at least one retaining strap includes an elastic retaining strap, fashioned as at least one piece connected at each sides of the shield portion.

9. An applicator according to claim 1, wherein said shield portion includes a rigid or semi-rigid transparent material.

10. An applicator according to claim 1, wherein a rim is provided on said shield portion, configured to facilitate a comfortable interface between said applicator and a socket around said user's eye.

11. An applicator according to claim 1, wherein said opening comprises two elongate channels in communication with each other.

12. An applicator according to claim 11, wherein said channels intersect at respective mid-points thereof.

13. An applicator according to claim 11, wherein said channels are substantially orthogonal relative to each other.

14. An applicator according to claim 1, wherein said support has a neck portion that is received in said opening.

15. An applicator according to claim 14, wherein said neck portion includes an outer circumferential groove with which contact edges of the opening engage.

16. An applicator according to claim 11, wherein in at least one of said channels has an enlarged opening therein.

17. An application according to claim 16, wherein said enlarged opening is located at an end of a channel.

18. An applicator according to claim 16, wherein said enlarged opening is shaped and configured to enable a circumferential groove of said support to pass therethrough.

19. An applicator according to claim 1, wherein an opening in said support contains means to regulate the flow of medication passing from said support to said user's eye.

20. An applicator according to claim 1, wherein said support is generally frusto-conical in shape, being tapered inwardly from an upper opening to a lower neck portion.

21. (canceled)
